(19) United States
(12) Patent Application Publication
(10) Pub. No.: US 2024/0060971 A1
(43) Pub. Date: Feb. 22, 2024

(54) SYSTEM AND METHOD FOR SIGNAL CALIBRATION IN A SENSOR SYSTEM

(71) Applicant: Siemens Healthineers Nederland B.V., Den Haag (NL)

(72) Inventor: Alexander van Reenen, Vught (NL)

(73) Assignee: Siemens Healthineers Nederland B.V., Den Haag (NL)

(21) Appl. No.: 18/267,449

(22) PCT Filed: Dec. 9, 2021
(86) PCT No.: PCT/EP2021/084960

§ 371 Date: Jun. 14, 2023

Related U.S. Application Data
(60) Provisional application No. 63/127,428, filed on Dec. 18, 2020.

(51) Int. Cl. G01N 33/543; G01N 21/82; G01N 21/27

(52) U.S. Cl. CPC G01N 33/54333; G01N 21/82; G01N 33/54306; G01N 21/274; G01N 21/552

(57) ABSTRACT

Methods and systems are provided for compensating for gravitational and fluid composition effects on a magnetic biosensor system. In one example, a sensor system includes a sample container configured to receive a sample containing an analyte to be tested, the sample container comprising a detection surface and a plurality of signal generating elements in the sample container, wherein the detection surface comprises a binding surface, which has been partially functionalized with capture elements that can bind, directly and/or indirectly, the analyte and/or the plurality of signal generating elements. The sensor system further includes a memory storing instructions executable by a processor to obtain background data comprising sensor signals from one or more background regions of the detection surface, obtain sample data comprising sensor signals from the binding surface, and perform a correction of the sample data based on the background data.

### FIELD OF TECHNOLOGY

The present description relates generally to systems and methods for a sensor system for the detection of an analyte in a sample, and more specifically to compensating for signal variations impacting the detection of analytes.

### BACKGROUND

Biosensors may allow for the detection of a given specific target molecule, referred to an analyte, within a sample, wherein the amount or concentration of said analyte is typically small, sometimes in the range of nanograms per milliliter. To detect these molecules, functionalized labels or detection tags, such as enzymes, fluorophores, or magnetic beads, may be utilized. In a magnetic-label biosensor, measuring the presence of the analyte (such as drugs or cardiac markers) is based on molecular capture and labeling with magnetic particles or beads. The magnetic beads may be arranged in a sample chamber of a sample cartridge. At least a portion of a sensor surface in the sample chamber is prepared for the detection of the analyte. For example, the sensor surface may include one or more regions where capture elements that are configured to bind to analyte are fixed (e.g., antibodies). For performing the test, a sample is loaded in the cartridge, and any analyte in the sample will bind both the magnetic beads and the capture elements on the binding surface.

Magnetic attraction of the beads, also referred to as actuation, may increase the performance, e.g., speed, of the biosensor for point-of-care applications. The direction of the magnetic attraction can be either towards the surface where the actual measurement is carried out or away from this surface. In the first case magnetic actuation allows the enhancement of the concentration of magnetic particles near the sensor surface (where the magnetic particles may bind to a corresponding capture element, such as an antibody, on the sensor surface via the analyte), speeding up the binding process of the magnetic particles at the sensor surface. In the second case, unbound magnetic particles (e.g., magnetic particles that are not bound to a capture element on the sensor surface) are removed from the surface which is called magnetic washing. Once magnetic washing is complete, the concentration of the analyte in the sample may be determined by measuring the number of magnetic beads bound to the capture elements on the sensor surface. For example, a light source may be directed to a region of the sensor surface where the capture elements are fixed so as to generate light that is totally internally reflected. The magnetic particles at the sensor surface may scatter and/or absorb the totally internally reflected light, which may be detected by a detector and used to determine the concentration of the target molecule in the sample.

### SUMMARY

In one embodiment, a sensor system includes a sample container configured to receive a sample containing an analyte to be tested, the sample container comprising a detection surface and a plurality of signal generating elements in the sample container, wherein the detection surface comprises a binding surface, which has been partially functionalized with capture elements that can bind, directly and/or indirectly, the analyte and/or the plurality of signal generating elements. The sensor system further includes a memory storing instructions executable by a processor to obtain background data comprising sensor signals from one or more background regions of the detection surface, obtain sample data comprising sensor signals from the binding surface, and perform a correction of the sample data based on the background data.

To the accomplishment of the foregoing and related ends, certain illustrative aspects of the system are described herein in connection with the following description and the attached drawings. The features, functions, and advantages that have been discussed can be achieved independently in various embodiments of the present disclosure or may be combined in yet other embodiments, further details of which can be seen with reference to the following description and drawings. The summary is provided to introduce a selection of concepts in a simplified form that are further described below in the Detailed Description. This summary is not intended to identify key features or essential features of any subject matter described herein.

### BRIEF DESCRIPTION OF THE DRAWINGS

FIG. 1 schematically shows a general setup of a sensor system according to the present disclosure.

FIGS. 2-4 schematically show an example sample cartridge of a sensor system having a plurality of binding surface areas according to the present disclosure.

FIG. 5 schematically shows the example sample cartridge of FIGS. 2-4 with a plurality of background regions positioned outside the binding surface areas.

FIG. 6 schematically shows an example signal generating element distribution within a sample cartridge according to the present disclosure.

FIG. 7 is a flow chart illustrating a method for testing a sample with a sensor system, using a background correction measured at the background regions outside the binding surface areas, according to the present disclosure.

FIGS. 8-12 show graphs illustrating various measurement parameters of an analyte using a sensor system when the background correction is or is not performed, according to the present disclosure.

FIG. 13 schematically shows the example sample cartridge of FIGS. 2-4 with a plurality of background regions positioned within the binding surface areas.

FIG. 14 is a flow chart illustrating a method for testing a sample with a sensor system, using a background correction measured at the background regions within the binding surface areas, according to the present disclosure.

### DETAILED DESCRIPTION

The following description relates to systems and methods for a sensor system, also referred to as a microfluidic testing system or microelectronic sensor system. The sensor system may be a magnetic sensor system including one or more sample containers loaded with functionalized signal generating elements, e.g., antibody-labeled magnetic particles, that are configured to bind to a specific target molecule (also referred to herein as an analyte or analyte of interest) such as troponin or B-type natriuretic peptide (BNP). Each sample container has a sensor detection surface that is also functionalized, e.g., with the same and/or different antibodies as those bound to the signal generating elements, to form a binding surface on the detection surface. To measure the concentration of the analyte in a sample such as blood or saliva, the sample is provided in the sample container, where the sample mixes with the signal generating elements. In this way, the signal generating elements may bind to the sensor binding surface via the analyte, with the number of signal generating elements that bind to the sensor binding surface being a function of the concentration of the analyte.

In examples where the sensor system is a magnetic sensor system, the signal generating elements may be magnetic particles and one or more magnetic elements may be positioned outside the sample container (e.g., below the sample container), and a magnetic field generated by the one or more magnetic elements may attract the magnetic particles to the sensor binding surface to expedite binding of the magnetic particle/analyte complexes to the sensor binding surface. The area of the sensor binding surface that binds the magnetic particle/analyte complexes may thus be based on the size and position of the magnetic element, as well any variations in the magnetic field generated by the magnetic element. Typically, the antibodies/capture elements are fixed to the sensor detection surface in discrete areas, such as discrete patches or spots. Further, some sample containers may be configured to facilitate detection of the concentration of more than one analyte, and thus different capture elements may be present in different binding surface areas. Accordingly, the positioning of the binding surface areas may be based on the magnetic field generated by the magnetic element. For example, if the magnetic field has a highest magnetic flux density at the center of the sensor detection surface, the binding surface may be positioned at the center of the sensor detection surface. In doing so, the magnetic elements may concentrate at and near the binding surface areas, which may increase the signal measured by the detector.

However, other forces also act on the signal generating elements, such as gravitational forces and/or forces generated during movement of the sensor system, which may influence the behavior of the signal generating elements during sample testing and thereby lead to result variations. Further, the mobility of the signal generating elements may be affected by the fluid of the sample being tested. For example, the viscosity of the sample fluid or the content of other substances in the sample fluid, such as sucrose or proteins, may influence the mobility of the signal generating elements as well as the optical signals produced by the signal generating elements that are measured to determine the concentration of the analyte.

As a result of the gravitational and/or other forces acting on the signal generating elements as well as the influence on signal generating element mobility from the sample fluid, signal generating element distribution may not be equal across the binding surface. This uneven signal generating element distribution may result in unreliable test measurements, particularly when more than one type of capture element is present on the sensor binding surface. Further, because the composition of the sample fluid may vary from sample to sample (e.g., some patients may have high blood sugar while other patients may have lower blood sugar), the influence on the optical signals of the signal generating elements by the fluid composition may result in test to test variations, which may also reduce test reliability.

Thus, according to embodiments disclosed herein, sample optical signals obtained during a detection phase of a test of a sample with a sensor system in order to measure the concentration of an analyte in the sample may be corrected with background optical signals obtained during a biochemical reaction phase that may precede the detection phase where the sample optical signals are obtained. The biochemical reaction phase may include periods where a magnetic element of the sensor system is activated to pull the magnetic particles to the sensor binding surface, and may occur prior to a final magnetic wash where the unbound magnetic beads are moved away from the sensor binding surface. By measuring the signal from the magnetic beads during the biochemical reaction phase where the magnetic beads are attracted to the sensor binding surface, the combined effects of the sample fluid properties and magnetic particle distribution inhomogeneities that can influence the sample optical signal response can be measured and used to directly correct the sample optical signal response measured at the binding surface (e.g., the sample optical signals). Further, the background optical signals may be obtained from a plurality of background regions of the detection surface that do not overlap with the binding surface areas (at least partially). In doing so, the background optical signals are not influenced by the analyte concentration, which influences the signal response within the binding surface areas during the biochemical reaction phase (e.g., during the biochemical reaction phase, the signal response will increase over time dependent on the analyte concentration). However, in some examples, the background regions may overlap the binding surface, and the presence of bound signal generating elements may be accounted for by subtracting out the sample data from the background data.

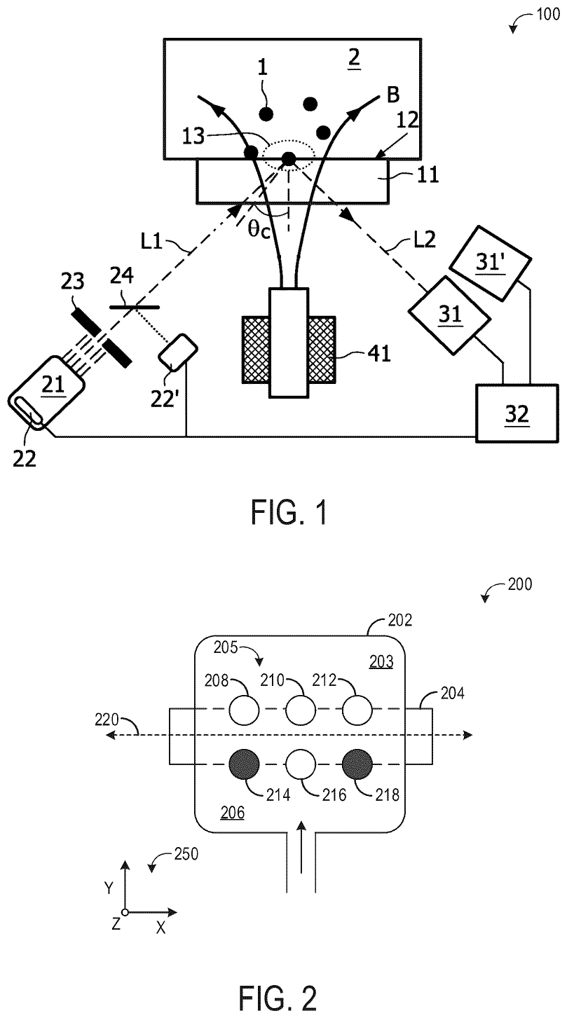

FIG. 1 schematically shows the general setup of a microelectronic sensor system 100 according to the present disclosure. The system 100 includes a carrier 11 that may for example be made from glass or transparent plastic like poly-styrene. The carrier 11 is located next to (e.g., below) a sample chamber 2 in which a sample fluid with target components to be detected (e.g. drugs, antibodies, DNA, etc.) can be provided. In some examples, the sample chamber 2 may be an interior area of a sample cartridge and the carrier 11 may form the bottom surface of the sample cartridge. In other examples, the sample chamber 2 may be an interior area of a microwell plate or other suitable container. The sample further comprises signal generating elements 1, for example superparamagnetic beads, wherein these elements 1 may be bound as labels to the aforementioned target components (for simplicity only the signal generating elements 1 are shown in FIG. 1).

The interface between the carrier 11 and the sample chamber 2 is formed by a surface referred to as a detection surface 12. This detection surface 12 may be coated with capture elements, e.g. antibodies, which can specifically bind the target components. Additional details regarding the coating of the detection surface 12 with capture elements is provided below.

The sensor system 100 comprises a magnetic field generator 41, for example an electromagnet with a coil and a core, for controllably generating a magnetic field B at the detection surface 12 and in the adjacent space of the sample chamber 2. With the help of this magnetic field B, the signal generating elements 1 can be manipulated, i.e. be magnetized and particularly be moved (if magnetic fields with gradients are used). Thus it is for example possible to attract signal generating elements 1 to the detection surface 12 in order to accelerate the binding of the associated target component to the detection surface 12.

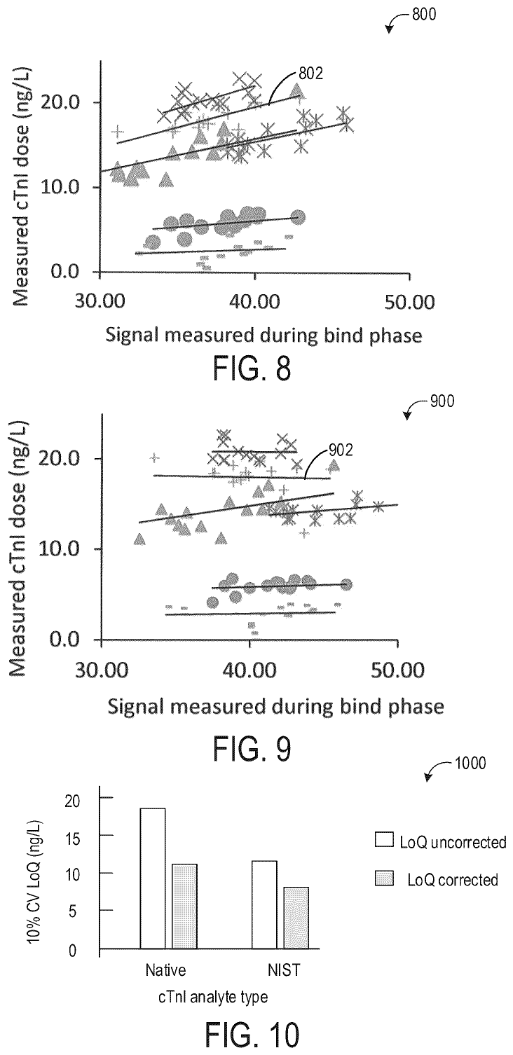

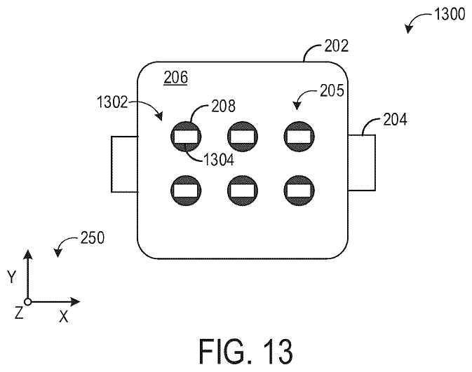

The sensor system 100 further comprises a light source 21, for example a laser or a light emitting diode (LED), that generates an input light beam L1 which is transmitted into the carrier 11. The input light beam L1 arrives at the detection surface 12 at an angle larger than the critical angle θc of total internal reflection (TIR) and is therefore totally internally reflected as an output light beam L2. The output light beam L2 leaves the carrier 11 through another surface and is detected by a light detector 31, e.g. a photodiode. The light detector 31 determines the amount of light of the output light beam L2 (e.g. expressed by the light intensity of this light beam in the whole spectrum or a certain part of the spectrum). The measurement results are evaluated and optionally monitored over an observation period by an evaluation and recording module 32 that is coupled to the detector 31. Module 32 may receive input data from the detector 31, process the input data, and output information for display on a display system and/or for storage (e.g., in a patient electronic medical record) in response to the processed input data, based on instruction or code programmed therein, corresponding to one or more routines. In particular, module 32 may be a microcomputer, including microprocessor unit, input/output ports, an electronic storage medium for executable programs and calibration values such as a read only memory chip, random access memory, keep alive memory, and a data bus. The storage medium read-only memory can be programmed with computer readable data representing instructions executable by the processor for performing the control methods for different components of FIG. 1, such as the methods described below with respect to FIGS. 7 and 13. Further, module 32 may be configured (e.g., execute instructions) to control the magnetic field generator 41 to provide a continuous or pulsed magnetic field when commanded, such as by controlling a current supply to the magnetic field generator 41.

In the light source 21, laser-diode (e.g., λ=658 nm) can be used. A collimator lens may be used to make the input light beam L1 parallel, and a pinhole 23 of e.g. 0.5 mm may be used to reduce the beam diameter. For accurate measurements, a highly stable light source is required. However, even with a perfectly stable power source, temperature changes in the laser can cause drifting and random changes in the output.

To address this issue, the light source may optionally have an integrated input light monitoring diode 22 for measuring the output level of the laser. The (low-pass filtered) output of the monitoring sensor 22 can then be coupled to the evaluation module 32, which can divide the (low-pass filtered) optical signal from the detector 31 by the output of the monitoring sensor 22. For an improved signal-to-noise ratio, the resulting signal may be time-averaged. The division eliminates the effect of laser output fluctuations due to power variations (no stabilized power source needed) as well as temperature drift (no precautions like Peltier elements needed).

In some examples, the final output of the light source 21 may be measured. As FIG. 1 coarsely illustrates, only a fraction of the laser output exits the pinhole 23. Only this fraction will be used for the actual measurement in the carrier 11, and is therefore the most direct source signal. Obviously, this fraction is related to the output of the laser, as determined by e.g. the integrated monitor diode 22, but will be affected by any mechanical change or instability in the light path (a laser beam profile is approximately elliptical with a Gaussian profile, i.e. quite non-uniform). Thus, it is advantageous to measure the amount of light of the input light beam L1 after the pinhole 23 and/or after eventual other optical components of the light source 21. This can be done in a number of ways. For example, a parallel glass plate 24 can be placed under 45° or a beam splitter cube (e.g. 90% transmission, 10% reflection) can be inserted into the light path behind the pinhole 23 to deflect a small fraction of the light beam towards a separate input-light monitoring sensor 22′. As another example, a small mirror at the edge of the pinhole 23 or the input light beam L1 can be used to deflect a small part of the beam towards a detector.

FIG. 1 includes a second light detector 31′ that can alternatively or additionally be used to detect fluorescence light emitted by fluorescent particles 1 which were stimulated by the evanescent wave of the input light beam L1. As this fluorescence light is usually emitted isotropically to all sides, the second detector 31′ can in principle be disposed anywhere, e.g. also above the detection surface 12. Moreover, it is of course possible to use the detector 31, too, for the sampling of fluorescence light, wherein the latter may for example spectrally be discriminated from reflected light L2.

As mentioned above, the sensor system may be configured to measure optical signals using total internal reflection (TIR). For example, the light source emits a light beam into the aforementioned carrier such that it is totally internally reflected in an investigation region at the detection surface of the carrier. The “investigation region” may be a sub-region of the detection surface or comprise the complete detection surface; it will typically have the shape of a substantially circular spot that is illuminated by the input light beam. Moreover, it should be noted that the occurrence of total internal reflection requires that the refractive index of the carrier is larger than the refractive index of the material adjacent to the detection surface. This is for example the case if the carrier is made from glass (n=1.6) and the adjacent material is water (n=1.3). It should further be noted that the term “total internal reflection” shall include the case called “frustrated total internal reflection” (fTIR), where some of the incident light is lost (absorbed, scattered etc.) during the reflection process.

By utilizing fTIR, the detection technique is surface-specific, which may reduce background noise. FTIR results in the generation of an evanescent wave in the sample, which decays exponentially away from the surface of the carrier. When this evanescent wave interacts with another medium like the signal generating elements 1 in the setup of FIG. 1, part of the incident light will be coupled into the sample fluid (this is called “frustrated total internal reflection”), and the reflected intensity will be reduced (while the reflected intensity will be 100% for a clean interface and no interaction). Depending on the amount of disturbance, i.e. the amount of signal generating elements on or very near (within about 200 nm) to the detection surface 12 (not in the rest of the sample chamber 2), the reflected intensity will drop accordingly. This intensity drop is a direct measure for the amount of bound signal generating elements 1, and therefore for the concentration of target molecules. When the mentioned interaction distance of the evanescent wave of about 200 nm is compared with the typical dimensions of anti-bodies, target molecules and magnetic beads, it is clear that the influence of the background will be minimal.

While FIG. 1 shows a microelectronic sensor system that uses an optical detection system to measure the concentration of analyte in the sample, other mechanisms for detecting the signal generating elements bound to the detection surface are possible. For example, the signal generating elements bound to the detection surface may be detected using magneto-resistive methods, hall-sensors, coils, optical methods, imaging, fluorescence, chemiluminescence, absorption, scattering, surface plasmon resonance, Raman, sonic detection, e.g. surface-acoustic-wave, bulk acoustic wave, cantilever, quartz crystal etc., electrical detection, e.g. conduction, impedance, amperometric, redox cycling, etc.

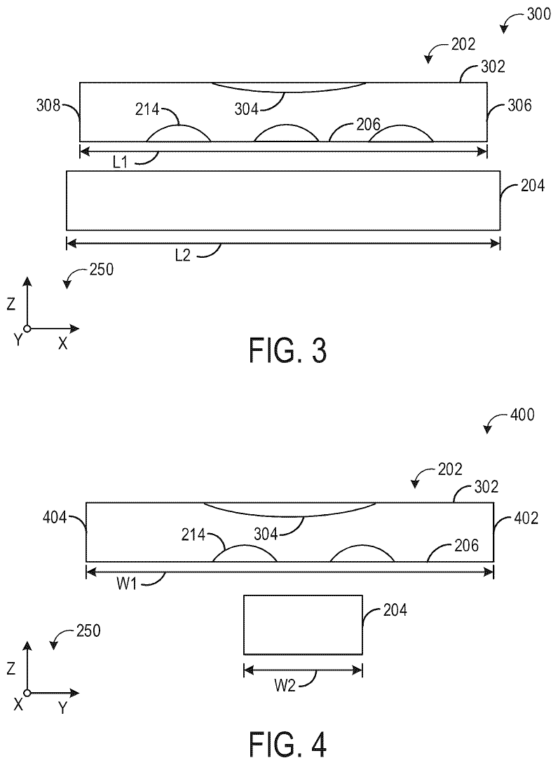

FIG. 2 schematically shows a top-down view 200 of an example sample cartridge 202 of a sensor system, such as the sample cartridge of the microelectronic sensor system 100 of FIG. 1. Sample cartridge 202 may include a plurality of walls and a hollow interior, thereby forming a sample chamber 203. Sample chamber 203 is a non-limiting example of sample chamber 2 of FIG. 1. During testing, a sample (liquid including one or more analytes of interest) is loaded into the sample cartridge 202 via an inlet, which may be along the arrow shown in FIG. 2. Sample cartridge 202 includes a binding surface 205 comprising capture elements (e.g., one or more antibodies) coated on a bottom surface of the sample cartridge 202 (the bottom surface of the sample cartridge 202 is also referred to as a detection surface 206). In the example shown, sample cartridge 202 includes six binding surface areas arranged into two rows. The binding surface 205 includes a first area 208, a second area 210, a third area 212 arranged in a first row, and a fourth area 214, a fifth area 216, and a sixth area 218 arranged in a second row. Each binding surface area may include the same capture element. For example, each binding surface area may include an anti-troponin antibody coated on the detection surface 206 at a given concentration. In other examples, one or more binding surface areas may include different capture elements. For example, half the binding surface areas may include an anti-troponin antibody and the other half of the binding surface areas may include an anti-BNP antibody. The portions of the detection surface 206 around and between the binding surface areas may not be functionalized with the capture elements that form/define the binding surface.

Also shown in FIG. 2 is a magnetic element 204, which may be included as part of the sensor system. The magnetic element 204 is a non-limiting example of magnetic field generator 41, and thus may include an electromagnet with a coil and a core, for controllably generating a magnetic field at the detection surface 206 and in the adjacent space of the sample chamber 203.

The magnetic element 204 may be configured to generate a magnetic field having a gradient, with the highest density of the gradient (e.g., the highest magnetic flux) extending along a magnetic axis, which in FIG. 2 may be a central axis 220 of the sample cartridge 202. In the example shown in FIG. 2, the central axis 220 may extend along (e.g., parallel to and aligned with) a longitudinal axis of the magnetic element 204. Further, FIG. 2 includes a Cartesian coordinate system 250, and the central axis 220 extends along (e.g., is parallel to) the X axis of the coordinate system 250.

FIGS. 3 and 4 show different views of the sample cartridge 202. FIG. 3 shows a first side view 300 and FIG. 4 shows a second side view 400 of the sample cartridge 202. Each of FIGS. 3 and 4 includes the Cartesian coordinate system 250. As shown in FIG. 3, the sample cartridge 202 includes a top wall 302, a first side 306, and a second side 308. Each of the first side 306 and second side 308 extends along the Z axis of the coordinate system 250, which may be parallel to gravity and point in a direction opposite of gravity (e.g., the positive Z direction is upward, away from flat ground). As shown in FIG. 4, the sample cartridge 202 also includes a third side 402 and a fourth side 404. Also shown in FIGS. 3 and 4 is a signal generating element region 304 where dried functionalized signal generating elements (e.g., magnetic beads) may be temporarily located. As shown, the signal generating element region 304 may be on an inner top surface (e.g., an inner surface of the top wall 302) of the sample cartridge 202, but other locations are possible and/or more than one signal generating element region may be included. When the sample is loaded into the sample cartridge 202, the dried functionalized signal generating elements may be released and mix with the sample.

As shown in FIG. 3, the sample cartridge 202 has a length L1 that extends from the first side 306 to the second side 308 along the X axis of the coordinate system 250. The magnetic element 204 has a length L2 that extends along the X axis, parallel to the longitudinal axis of the magnetic element 204. In the example shown, the length L2 of the magnetic element 204 may be as long or longer than the length L1 of the sample cartridge 202.

As shown in FIG. 4, the sample cartridge 202 has a width W1 that extends from the third side 402 to the fourth side 404 along the Y axis. In some examples, the width W1 of the sample cartridge 202 may be equal to the length L1 of the sample cartridge 202. In other examples, the width W1 may be longer or shorter than the length L1. The magnetic element 204 has a width W2 that extends along the Y axis, perpendicular to the longitudinal axis of the magnetic element 204. In the example shown, the width W2 of the magnetic element 204 is shorter than width W1 of the sample cartridge 202. Further, the magnetic element 204 may be centered with respect to the sample cartridge 202, such that a central longitudinal axis of the magnetic element 204 is aligned with a central axis of the sample cartridge 202, where the central axis of the sample cartridge may be positioned at a point equidistant between the third side 402 and the fourth side 404 and extend from the first side 306 to the second side 308. In this way, the central longitudinal axis of the magnetic element 204 may be positioned between the two rows of binding surface areas.

The magnetic element 204 causes magnetic field gradients toward the center of the sample cartridge, e.g., along the central axis 220. Because the magnetic element 204 has a longer length L2 than the length L1 of the sample cartridge 202, the magnetic field gradient may be consistent along the length L1 of the sample cartridge 202 but may vary along the width W1 of the sample cartridge 202. For example, along the central axis 220, the magnetic field may have a highest flux density along an entirety of the central axis 220 from the first side 306 to the second side 308. However, the magnetic flux density may decrease from the central axis 220 to the third side 402 and from the central axis 220 to the fourth side 404.

Thus, a magnetic field is generated when the magnetic element 204 is activated (e.g., current is supplied to the coil of the magnetic element 204). The magnetic field may have a gradient with a region of highest magnetic flux density positioned along the center of the sample cartridge, e.g., the central axis 220. After the sample is loaded into the sample cartridge 202, the signal generating elements are released and mix with the sample. When the magnetic element 204 is activated, the signal generating elements (which may be magnetic particles) and any bound analyte will be pulled to the detection surface 206 by magnetic force, and particularly be pulled toward the central axis 220, where the signal generating elements will interact with the capture elements fixed to the detection surface 206 (e.g., as the binding surface 205). Thus, the signal generating elements dispersed in the sample will concentrate toward the location of the highest field line density/highest magnetic flux density.

Accordingly, to ensure consistent analyte analysis, particularly when more than one analyte is being tested, the binding surface 205 may be positioned at or in proximity to the location of the highest field line density/flux density. For example, referring back to FIG. 2, the binding surface 205 is arranged in proximity to the central axis 220 (e.g., each area may be arranged above the magnetic element and within a threshold distance from the central axis 220). In this way, when the signal generating elements are concentrated at the detection surface 206 via a magnetic field generated by the magnetic element, the signal generating elements will concentrate at and along the binding surface 205, which will increase the likelihood that any magnetic particle/analyte complexes will interact with and bind the appropriate antibodies forming the binding surface 205.

While in the example shown in FIGS. 2-4, the sample cartridge 202 includes six binding surface areas and is positioned over a single magnetic element with a magnetic axis aligned with a central axis of the sample cartridge 202, other configurations are possible without departing from the scope of this disclosure. For example, more or fewer binding surface areas may be included, such as a single binding surface area, two binding surface areas, three binding surface areas, etc. The binding surface areas may be arranged differently than shown in FIGS. 2-4, such as arranged in a single row, arranged in three rows, arranged in a circle, etc. The sensor system may include more than one magnetic element in some examples. Further, in some examples, the magnetic element may generate a magnetic field with a highest magnetic flux density centered at a single point rather than along an axis.

The sample cartridge 202 is configured to be positioned in a sensor system, such as the sensor system of FIG. 1. The sample cartridge 202 includes reagents for carrying out a test, e.g., to measure the concentration of an analyte in a sample. The reagents may include functionalized magnetic particles (e.g., magnetic particles including a capture element specific to the analyte, such as the signal generating elements contained in the signal generating element region 304) and the binding surface 205, and may additionally include buffers or other reagents. The sample, such as blood or saliva, is introduced into the sample chamber of the sample cartridge, where the sample mixes with the magnetic particles and the capture elements bound to the detection surface 206. When the sample is introduced into the sample chamber and the signal generating elements are dispersed, a biochemical reaction phase of the test commences. During the biochemical reaction phase, the analyte in the sample (e.g., troponin) binds to the functionalized signal generating elements and/or to the binding surface. In the biochemical reaction phase, the analyte molecules may be in four states: unbound, bound only to a signal generating element, bound only to the binding surface, or bound to both the binding surface and a signal generating element. To expedite the process of the analyte reaching the fourth state (where the analyte is bound to both the binding surface and a signal generating element), the magnetic element may be activated in order to generate a magnetic field that actively brings the signal generating elements near the binding surface of the sample cartridge.

After a threshold amount of time has elapsed, the biochemical reaction phase is stopped by applying a magnetic field that pulls unbound signal generating elements (e.g., signal generating elements that are not bound to the binding surface via the analyte and/or a capture element) away from the binding surface. Upon this magnetic washing, a detection phase commences, where optical fields are used to obtain a measurement of the number of signal generating elements that remain at the binding surface (due to the signal generating elements being bound to the binding surface). The optical signals measured during the detection phase, which may be referred to herein as sample data, are then compared to calibration information that may be predetermined and obtained from a tag on the sample cartridge, such as an RFID tag, in order to calculate the concentration of the analyte. Typically, the optical signals used to calculate the concentration of the analyte are obtained from specific pre-defined measurement regions of interest (ROIs) on the binding surface. These measurement ROIs may be subsets of the binding surface, e.g., rectangular areas that overlap the areas of the binding surface. Each measurement ROI may alternatively be the complete area of a respective binding surface area, or an area that includes a respective binding surface area as well as some area outside the respective binding surface area.

The testing process described above may result in variations in the optical signals detected at each measurement ROI and/or test-to-test optical signal variations due signal generating element distribution inhomogeneity and/or differing sample fluid parameters, such as viscosity. For example, a first patient being tested for an analyte concentration (e.g. troponin) may submit a sample (e.g., blood) having higher blood sugar content relative to a sample from a second patient. The higher sugar content may cause the signal generating elements in the sample from the first patient to have optical properties that differ from the signal generating elements in the sample from the second patient, as the optical properties of the signal generating elements are influenced by the material composition of the signal generating elements and the fluid surrounding the signal generating elements. In this way, the test results between the first patient and the second patient may vary due to the blood sugar content of the patients' blood samples, in addition to different levels of the analyte. Further, in some examples, different patient samples may exhibit different fluid viscosity, which may impact signal generating element mobility during testing, which may result in testing variations.

To address the above-described issues of signal generating element distribution inhomogeneities and different sample fluid properties, background data may be obtained during the biochemical reaction phase of the test (e.g., before applying the magnetic field to remove the unbound magnetic particles from the binding surface) or at another suitable time of the test and used to correct the sample data obtained during the detection phase. The background data may include optical signals that are obtained when bound and unbound magnetic particles are present at the binding surface, and thus may be obtained when the magnetic field is applied to actively pull the magnetic particles to the binding surface, but also could be obtained when magnetic fields are not being applied.

However, to further improve the correction of the sample data with the background data, the background data may include the measurement of optical signals from only unbound signal generating elements, as the concentration of the analyte will influence the binding of the signal generating elements to the binding surface. Thus, the background data may be obtained from background regions that do not overlap the binding surface.

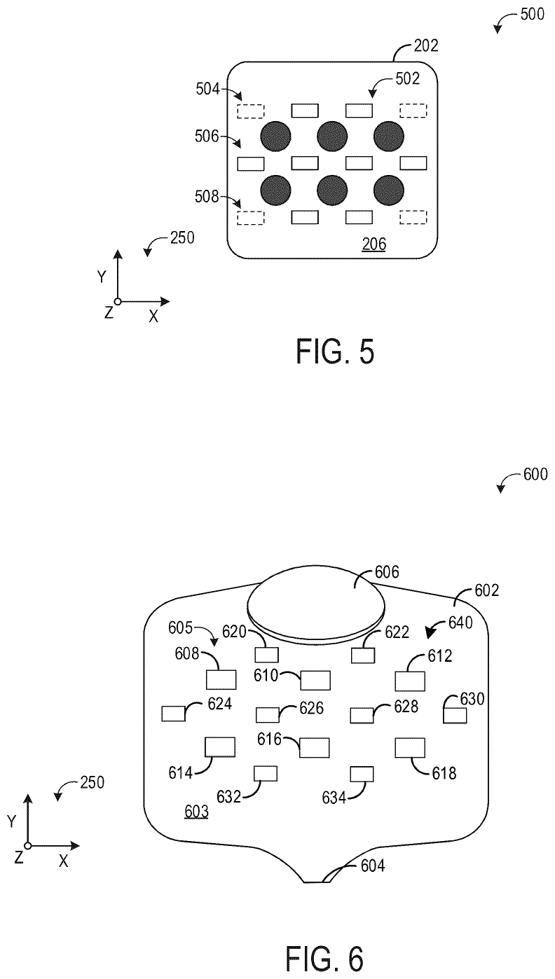

FIG. 5 shows another schematic view 500 of the sample cartridge 202 including the positions of a plurality of background regions 502. In the example shown, the plurality of background regions 502 are arranged to surround each discrete binding surface area, and thus are arranged into a first row 504, a second row 506, and a third row 508. In some examples, each row may include four background regions for a total of 12 background regions. In the example shown, the first row 504 includes background regions numbered 1-4 (left to right), the second row 506 includes background regions numbered 5-8, and the third row 508 includes background regions numbered 9-12. It is to be appreciated that the background regions are regions of the detection surface 206 where optical signals are to be detected to generate the background data as described above, and no capture elements may be fixed to the detection surface at the background regions. In some examples, the corner background regions (shown in dashed lines in FIG. 5) may be dispensed with. As used herein, the term “background region” may refer to an area (which may be circular, rectangular, or of another suitable shape) of a detection surface of a sample cartridge where sensor signals (such as optical signals) are measured to generate background data used to correct sample data, where the sample data is used to determine the concentration of one or more analytes in a sample. In some examples, the background region(s) may fully or partially overlap with the binding surface. In other examples, the background region(s) may not overlap with the binding surface. The sensor signals measured at the background region(s) as described herein may include optical signals or other types of signals (e.g., magnetic) generated by signal generating elements (which may be magnetic particles or other types of particles or beads that are able to be optically or otherwise detected), which may be bound to the detection surface (e.g., in areas where the detection surface is coated with one or more capture elements) and/or unbound to the detection surface. In examples where fTIR is used to generate and measure the optical signals, the optical signals output by the signal generating elements within a threshold range of the detection surface (e.g., within 100 nm) may be measured, while signal generating elements outside of the threshold range are not detected.

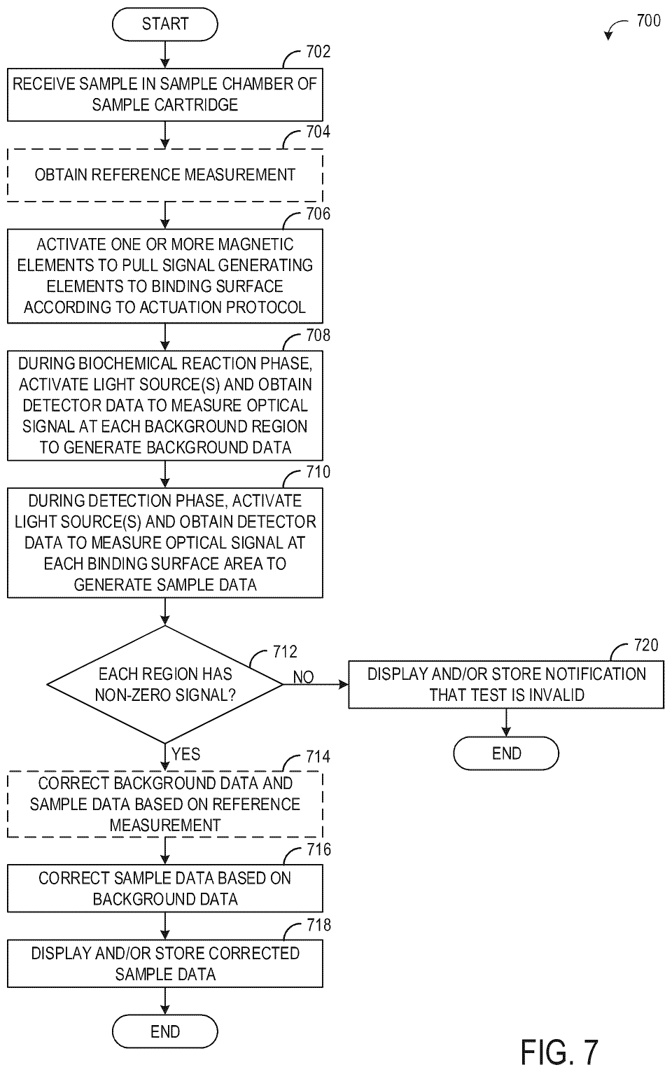

FIG. 6 shows an example image 600 of a sample cartridge 602 after a sample has been introduced into the sample cartridge and during the biochemical reaction phase where signal generating elements 640 have mixed with the sample and are being pulled to a binding surface 605 on a detection surface 603 of the sample cartridge. The sample cartridge 602 is a non-limiting example of the sample cartridge 202 explained above, and thus includes an inlet 604 at one end of the detection surface 603 and a pinning 606 at another, opposite end of the detection surface 603. The binding surface 605 includes six discrete areas of capture elements fixed to the detection surface 603, where the areas are arranged into two rows, including a first area 608, a second area 610, a third area 612, a fourth area 614, a fifth area 616, and a sixth area 618. Each area of the binding surface 605 is represented by a rectangle that may indicate a measurement ROI (e.g., where the optical signals from the magnetic particles at the binding surface will be detected). However, other shapes for the areas of capture elements and/or measurement ROIs are possible without departing from the scope of this disclosure.

FIG. 6 further illustrates the location of a plurality of background regions, which as shown are arranged into three rows. The first (top) row of background regions includes a first background region 620 and a second background region 622. The second (middle) row of background regions includes a third background region 624, a fourth background region 626, a fifth background region 628, and a sixth background region 630. The third (bottom) row of background regions includes a seventh background region 632 and an eighth background region 634.

The dark spots/streaks in FIG. 6 are indicative of signal generating elements 640. The signal generating element distribution during the biochemical reaction phase varies across the detection surface 603. For example, due to the configuration of the magnetic element (not shown in FIG. 6), the signal generating elements 640 are concentrating along a center region of the detection surface and no (or few) signal generating elements are present along the detection surface near the top (e.g., including the pinning 606) or bottom (e.g., including the inlet 604) of the sample cartridge. The background regions are positioned to exclude these zero density areas and are further positioned to surround the areas of the binding surface.

In the example shown in FIG. 6, the signal generating elements are exhibiting a non-uniform distribution. For example, more signal generating elements are present on the left side and on the right side of the sample cartridge 602 than in the center of the sample cartridge 602. As such, the optical signals obtained during the detection phase may be stronger for the areas of the binding surface on the left and on the right than in the center of the sample cartridge. Thus, the background data may be used to correct the sample data. For example, the optical signals measured at the background regions surrounding the first area 608 (e.g., the background regions 620, 624, and 626) may be combined (e.g., averaged) and used to correct the sample data obtained at the first area 608. In other examples, the optical signals measured at each background region may be combined to form an overall background dataset that may be used to correct the sample data obtained at each area of the binding surface. In this way, the optical signals measured during the biochemical reaction phase are considered as a calibration measurement of the optical signal capability of the specific test and thus used to correct the sample data. In doing so, variations in signal generating element concentrations or fluid properties such as refractive index or viscosity may be compensated.

While a sample cartridge configured to be positioned in a sensor device or sensor system has been described herein, it is to be appreciated that the sample cartridge may be any suitable container that is coated with one or more capture elements to form a binding surface thereof and configured to house a sample mixed with signal generating elements. For example, the sample cartridge may not be enclosed as described herein but may instead lack a top wall, or the sample cartridge may be in the form a plate including one or more wells. As such, the sample cartridge(s) described above with respect to FIGS. 2-6 may be referred to as a sample container, which may include a cartridge, a plate, a multi-well plate, or virtually any other structure capable of housing a sample and having a binding surface as described herein.

FIG. 7 is a flow chart illustrating a method 700 for testing a sample with a sensor system, such as sensor system 100, including applying a background correction using background data collected at a plurality of background regions that do not overlap any areas of a binding surface (such as the plurality of background regions shown in FIG. 5 and/or FIG. 6). Method 700 may be executed at least partially by a computing system such as evaluation and recording module 32 of sensor system 100, according to instructions stored in memory thereof that are executed by a processor. At 702, a sample is received in a sample chamber of the sensor system. The sample may include a biological fluid, such as blood, saliva, etc., which may be mixed with reagents, buffers, water, etc. The sample may be introduced via a sample inlet and may flow into the sample chamber. The sample chamber may comprise an interior of a sample container, such as sample cartridge 202. Thus, the sample may mix with signal generating elements (such as magnetic particles) in the sample chamber. The sample container may include one or more types of capture elements coated on a detection surface of the sample container, thereby forming a binding surface.

At 704, a reference measurement is optionally obtained. Obtaining the reference measurement may include activating one or more light sources of the sensor system and detecting the resultant optical signals with one or more detectors of the sensor system. The reference measurement may be obtained prior to the commencement of the biochemical reaction phase, e.g., prior to activation of a magnetic element of the sensor system. At 706, one or more magnetic elements of the sensor system are activated in order to attract the signal generating elements to the binding surface of the sample cartridge. The one or more magnetic elements may include one or more magnetic elements, such as magnetic element 204, that generate a magnetic field centered along a magnetic axis or a single point. The magnetic element(s) may be activated to generate a continuous or a pulsed magnetic field, according to a predetermined actuation protocol.

At 708, during the biochemical reaction phase where the one or more magnetic elements are being actuated according to the actuation protocol, one or more light sources of the sensor system are activated, such as light source 21, and detector data is obtained from one or more detectors, such as detector 31, to measure the optical signals at each background region of the sample container in order to generate background data. For example, the light source(s) positioned to direct light to the background regions may be activated and the resultant optical signals may be measured by corresponding detector(s). The collection of the background data may be timed to correspond with actuation periods where the magnetic field is being applied to pull the signal generating elements to the binding surface, at least in some examples. The optical signals may be obtained at one or more discrete time points during the biochemical reaction phase, or the optical signals may be obtained continuously during the biochemical reaction phase. In some examples, the optical signals may be obtained using FTIR based detection. In such examples, only the (magnetic) particles are detected that are in close proximity of the detection surface, e.g., within the evanescent wave which typically penetrates in the sample chamber by about 100 nm. Additionally, when using pulsed magnetic fields, the magnetic particles will not be close to the detection surface for the whole duration of a pulse (when the magnetic field is switched off for several 100 ms or seconds), and the measured signals thereby correspond to the effective time in which particles are at the detection surface (and can only then bind to the binding surface).

At 710, during the detection phase that commences after at least one magnetic wash has been performed (e.g., where a magnetic wash includes a magnetic field being applied to move unbound signal generating elements away from the binding surface of the sample container), one or more light sources are activated and detector data is obtained to measure the optical signals at each area of the binding surface in order to generate sample data. For example, the light source(s) positioned to direct light to the binding surface may be activated and the resultant optical signals may be measured by corresponding detector(s). The collection of the sample data may be performed only once the biochemical reaction phase is complete, in some examples. In other examples, the sample data may be collected at multiple time points during interruptions in the biochemical reaction phase. For example, the biochemical reaction phase may be paused so that a first sample dataset may be collected (after a magnetic wash is performed), and then the biochemical reaction phase may be resumed. Then the biochemical reaction phase may be terminated and a second sample dataset may be collected (after another magnetic wash is performed). By collecting sample data at one or more time points before the biochemical reaction phase is completed, signal saturation due to high analyte concentration, for example, may be avoided by measuring the optical signals before the biochemical reaction has gone to completion. The optical signals may be obtained at one or more discrete time points during the detection phase, or the optical signals may be obtained continuously during the detection phase. The timing of when the optical signals are obtained may be based on a desired signal to noise ratio of the signals (e.g., signals obtained closer to when the binding surface is saturated may have a higher signal to noise ratio) and/or a desired speed of performing the test. Further, similar to the background optical signals, the optical signals obtained to generate the sample data may be obtained using fTIR.

At 712, method 700 determines if the optical signals measured from each background region are non-zero signals. For example, the signal response detected from each background region during the collection of the background data may be analyzed to confirm that each region registered a positive, non-zero value from the output of the corresponding detector(s). Given the density of magnetic particles, at least some signal is expected to be measured from each background region. If no signal is detected (e.g., a zero value, or within a threshold range of zero) from one or more background regions, it may be indicative of an air bubble or otherwise a lack of the sample completely filling the sample container, which may compromise the test results. Thus, if one or more background regions register a signal of zero or register a signal within a threshold range of zero (e.g., the answer at 712 is NO), method 700 proceeds to 720 to display and/or store a notification that the current test is invalid and/or that the analyte concentration cannot be determined, and then method 700 returns.

However, if each background region has a positive, non-zero signal (e.g., the answer at 712 is YES), method 700 proceeds to 714 where the background data and the sample data are optionally corrected based on the reference measurement obtained at 704. For example, the reference measurement may be subtracted from each of the background data and the sample data. In doing so, other fluctuations that may affect the optical signal (e.g., output from the light sources) may be compensated. At 716, the sample data is corrected based on the background data. As explained previously, the background data from one or more background regions positioned adjacent or surrounding an area of the binding surface may be combined and used to correct the sample data for that area of the binding surface. In other examples, the background data from all the background regions may be combined and collectively used to correct the sample data from each area of the binding surface. Correcting the sample data based on the background data may include dividing the sample data by the background data. In other examples, a different function may be applied to correct the sample data using the background data, such as a relation between the sample data and the background data that is established during calibration, where the relation may be linear, a power function, exponential, etc.

In some examples, the background data may be weighted so that optical signals obtained from some background regions are given a higher weight than optical signals obtained from other background regions. For example, referring to FIG. 6, the optical signals from the second (middle) row of background regions (background regions 624, 626, 628, and 630) may be given a lower weight than the optical signals from the first (top) and third (bottom) rows of background regions. After weighting, the optical signals may be combined (e.g., summed or averaged) to generate the background data. By giving the background regions in the middle row a lower weight, the tendency for the signal generating elements to concentrate along the middle of the sample cartridge where the highest magnetic flux density is located may be compensated.

At 718, the corrected sample data may be stored and/or displayed on a display of the sensor system. The corrected sample data may be used to determine a concentration of one or more analytes of interest in the sample, and the determined concentration(s) or concentration signal(s) may be output for display and/or saved in memory. For example, the computing system may access a relationship between the corrected sample data and a concentration of an analyte (e.g., from an RFID tag on the sample container, from a relationship stored in memory, etc.) and determine the concentration of the analyte based on the corrected sample data and the relationship. For example, the concentration of the analyte may be computed using a calibration curve to convert the measured amount of bound signal generating elements into a concentration of the analyte. The calibration curve (or formula, or equation) may be stored in the memory of the sensor system (e.g., the evaluation and recording module 32), and the values/parameters for the calibration curve or formula may be stored onto an RFID tag of the sensor system. The calibration parameters (e.g., the calibration curve or formula, including constants of the formula) may be determined after manufacturing by testing a series of cartridges with reference samples, e.g., samples containing different concentrations of analyte, distributed over the reportable range for the test. The test data is then subsequently analyzed by fitting (e.g., a least squares regression) the data using a mathematical formula. The resulting fit parameters are then written onto the RFID tag of the device. Method 700 then ends.

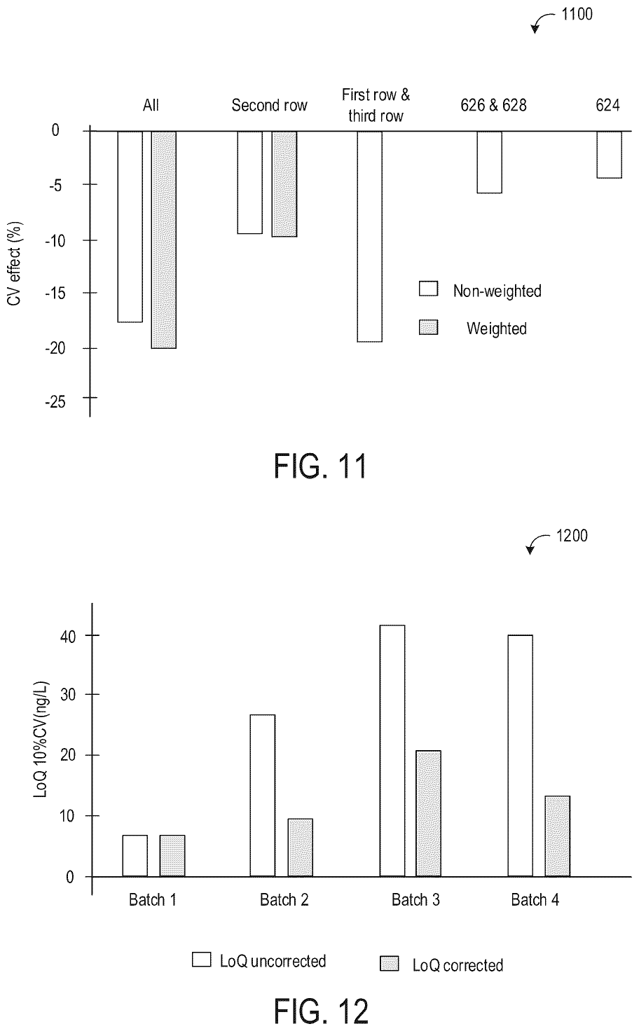

FIGS. 8-10 show example graphs demonstrating the effect of the background correction described above with respect to FIG. 7. For each graph, sample data and/or background data were obtained on a plurality of different samples from different patients, with each sample spiked with a different amount of an analyte, herein troponin-I. The samples were measured using a sensor system, such as the sensor system of FIG. 1. The sample containers used to measure the samples may be the sample cartridge of FIGS. 2-6, which in the example shown may include a binding surface arranged into six discrete areas as shown in FIG. 2 with each area of the binding surface including an anti-troponin antibody. In the examples shown, six samples were measured and each sample was measured 15 times. For each sample measurement, a concentration of troponin was determined during a respective detection phase. Further, for each sample measurement, background data was obtained during the biochemical reaction phase, at each background region, as described in more detail below.

FIG. 8 shows a graph 800 of measured troponin concentration as a function of an average optical signal measured during the biochemical reaction phase (also referred to as a bind phase) for each sample. Thus, the y-axis of graph 800 is measured troponin-I concentration (cTnI) in ng/L and the x-axis shows the average optical signal obtained during the biochemical reaction phase (which is normalized to a reference measurement and thus is a percentage relative to the reference measurement). The graph 800 of FIG. 8 illustrates the measured troponin concentration as based on optical signals obtained during the detection phase, without performing a background correction. Each measured troponin concentration for a given sample (e.g., 13-15 measured troponin concentrations) was plotted as a function of the average optical signal during the biochemical reaction phase for that measurement. For example, line 802 is a best-fit line of 13 measured troponin concentrations for a first sample during respective detection phases as a function of an average optical signal measured during the biochemical reaction phase for a given sample. Each individual troponin concentration measurement of the first sample is shown in FIG. 8 as a plus symbol plotted as a function of the average optical signal measured during the biochemical reaction phase. As appreciated from graph 800, the measured troponin concentration increases as the average optical signal detected during the biochemical phase increases, as demonstrated by the increasing slope of line 802 and the rest of the tested samples (each tested sample is shown as a best-fit line with the corresponding individual measurements shown as various symbols). The correlation between the measured analyte concentration and the optical signals indicates a potential coefficient of variation (CV) effect. In other words, each sample has a known troponin concentration that should be measured during each measurement. However, test-to-test variations in magnetic particle binding (e.g., due to non-even particle distribution, sample fluid properties, etc.) can result in artificially low or artificially high troponin concentration measurements. For example, the measurements of troponin concentration in the first sample show a relatively high level of variation, spanning from approximately 15 ng/L to 20 ng/L.

FIG. 9 shows a graph 900 of measured troponin concentration of the plurality of samples as a function of the optical signal measured during the biochemical reaction phase for each sample, with the background correction applied. Thus, the y-axis of graph 900 is measured troponin-l concentration (cTnl) in ng/L and the x-axis shows the optical signal obtained during the biochemical reaction phase. The samples measured to generate the graph 900 are the same as the samples measured to generate the graph 800, but in graph 900, the measured troponin concentration was determined based on the optical signals obtained during the detection phase with the optical signals corrected using the background correction described herein (e.g., as described above with respect to FIG. 7). The specific background correction applied to generate graph 900 included measuring the optical signals at the background regions shown in FIG. 6. The optical signals from the first row of the background regions and the third row of the background regions were weighted relative to the optical signals of the second row of the background regions. The total weighting to the background regions was (from left to right then up to down) of factors 2, 2, 1, 1, 1, 1, 2, and 2. The measured optical signals from the plurality of areas of the binding surface (e.g., the sample data) were divided by the measured optical signals at the background regions (e.g., the background data) during the biochemical reaction phase and multiplied by a factor of 37.5 (which was the average optical signal of the background data overall).

As appreciated by FIG. 9, the observed correlation between the measured analyte concentration and the optical signals were reduced for each sample. For example, line 902 shows a best-fit line of the measured troponin concentration for the first sample as a function of an averaged, corrected optical signal measured during the biochemical reaction phase for the given sample (corresponding to the sample measured to generate the line 802). The line 902 shows a reduced correlation between the measured analyte concentration and the optical signals during the biochemical reaction phase. Thus, by correcting the sample data with background data indicative of the overall magnetic particle binding, test-to-test variations in magnetic particle binding may be accounted for, increasing accuracy and reproducibility of the troponin concentration measurement.

The limit of quantification (LoQ) 10% coefficient of variation (CV) (LoQ10% CV) was calculated for the uncorrected troponin concentration measurements and the corrected troponin concentration measurements and plotted as shown in FIG. 10. Graph 1000 of FIG. 10 shows the LoQ10% CV for measured troponin concentration (in ng/L) for two types of troponin (native and a reference troponin developed by the NIST), when uncorrected or corrected as described above. As appreciated from FIG. 10, the background correction lowers the LoQ for each type of troponin.

FIG. 11 shows a graph 1100 illustrating the effect on the CV of using different combinations of background regions. The different combination of background regions shown in FIG. 11 include all the background regions (e.g., the first, second, and third rows shown in FIG. 6), only the second row of background regions, only the first row and the third row of background regions, only the center-most two background regions (the background regions numbered 626 and 628 in FIG. 6), and only a single background region of the second row (the background region numbered 624 in FIG. 6). Each background region combination showed an effect on the CV. For example, measuring the optical signals from each background region showed an 18% reduction on the CV (the column labeled “All”). Further, in some of the combinations, the background regions were also weighted, so that the second row of background regions were weighted with a factor of 1 and the first and third rows of background regions were weighted with a factor of 4. This weighting showed an improvement in the CV effect relative to an unweighted background correction, such as an improvement of an 18% reduction of the CV to a 21% reduction of the CV. Graph 1100 also shows that the background regions near the pinning and the inlet of the sample container (e.g., the first row and the third row of the background regions) contributed the most to the CV improvement after correction.

The improvement of the measured troponin concentration CV and LoQ10% CV due to the background correction was tested on additional batches of sample containers, as shown by graph 1200 of FIG. 12. The batches may vary in casein concentration, bloodhousing, or other factors, but all tested batches included six anti-troponin antibody spots. Graph 1200 shows that while the effect of the background correction on the LoQ10% CV varied from batch to batch, the background correction showed a decrease in the LoQ for each batch other than batch 1, thereby showing a high reproducibility in the effect of the background correction.

FIG. 13 shows another background region layout that may be used to obtain the background data for correcting the sample data in order to perform a background correction and reduce test-to-test variability in analyte concentration measurement. In FIG. 13, a schematic view 1300 of the sample cartridge 202 is shown in including the positions of a plurality of background regions 1302. In the example shown in FIG. 13, the plurality of background regions 1302 is arranged so that each background region overlaps a corresponding area of the binding surface. For example, a first background region 1304 is positioned at the same location as the first area 208, and each remaining background region is positioned at a same location as a different area of the binding surface (such that six background regions are included, one positioned at each area of the binding surface that is functionalized to include capture elements). It is to be appreciated that the background regions are regions of the detection surface 206 where optical signals are to be detected during the biochemical reaction phase as described above.

When the background regions are positioned as shown in FIG. 13, the optical signals measured during the biochemical reaction phase include signals from both bound and unbound magnetic particles. Thus, the signals from only the unbound magnetic particles can be obtained (and used as the background data to correct the sample data as described above) by using the optical signals obtained when the biochemical reaction phase is complete. For example, the optical signals after the biochemical phase is complete (e.g., the optical signals obtained during the detection phase) are subtracted from the optical signals obtained during the biochemical reaction phase to obtain the background data.

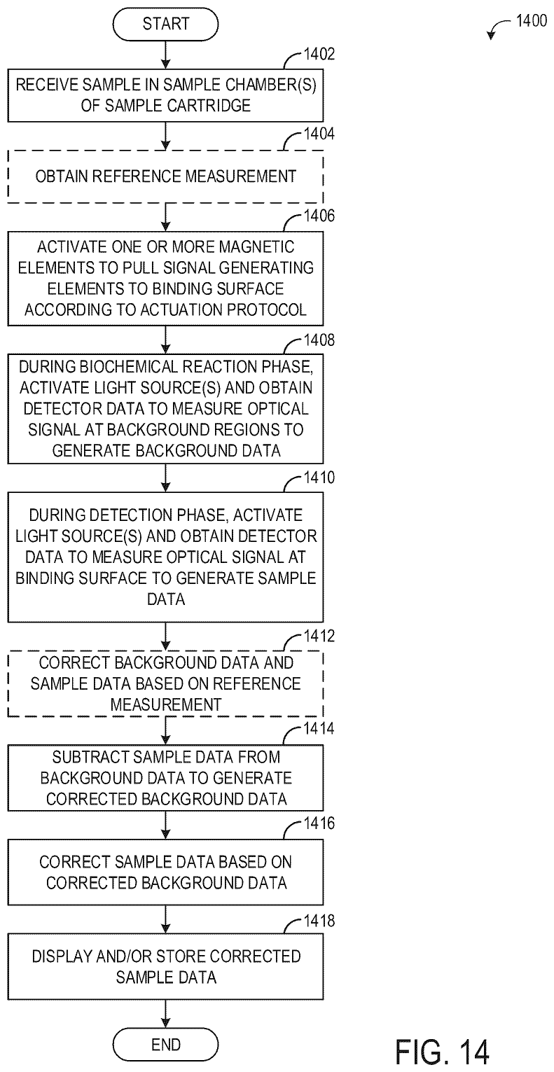

FIG. 14 is a flow chart illustrating a method 1400 for testing a sample with a sensor system, such as sensor system 100, including applying a background correction using background data collected at a plurality of background regions that each overlap a respective binding surface area (such as the plurality of background regions shown in FIG. 13). Method 1400 may be executed at least partially by a computing system such as evaluation and recording module 32 of sensor system 100, according to instructions stored in memory thereof. At 1402, a sample is received in a sample chamber of the sensor system. The sample may include a biological fluid, such as blood, saliva, etc., which may be mixed with reagents, buffers, water, etc. The sample may be introduced via a sample inlet and may flow into the sample chamber. The sample chamber may comprise an interior of a sample container, such as sample cartridge 202. Thus, the sample may mix with signal generating elements (e.g., magnetic particles) in the sample chamber. The sample container may include one or more types of capture elements coated on a detection surface of the sample container, thereby forming a binding surface.

At 1404, a reference measurement is optionally obtained. Obtaining the reference measurement may include activating one or more light sources of the sensor system and detecting the resultant optical signals with one or more detectors of the sensor system. The reference measurement may be obtained prior to the commencement of the biochemical reaction phase, e.g., prior to activation of a magnetic element of the sensor system. At 1406, one or more magnetic elements of the sensor system are activated in order to attract the signal generating elements to the binding surface of the sample cartridge. The one or more magnetic elements may include one or more magnetic elements, such as magnetic element 204, that generate a magnetic field centered along a magnetic axis or a single point. The magnetic element(s) may be activated to generate a continuous or a pulsed magnetic field, according to a predetermined actuation protocol.

At 1408, during the biochemical reaction phase where the one or more magnetic elements are being actuated according to the actuation protocol, one or more light sources of the sensor system are activated, such as light source 21, and detector data is obtained from one or more detectors, such as detector 31, to measure the optical signals at each background region of the sample container in order to generate background data. For example, the light source(s) positioned to direct light to the background regions may be activated and the resultant optical signals may be measured by corresponding detector(s). The collection of the background data may be timed to correspond with actuation periods where the magnetic field is being applied to pull the signal generating elements to the binding surface, at least in some examples.

At 1410, during the detection phase that commences after at least one magnetic wash has been performed (e.g., where a magnetic wash includes a magnetic field being applied to move unbound magnetic particles away from the binding surface of the sample container), one or more light sources are activated and detector data is obtained to measure the optical signals at each area of the binding surface in order to generate sample data. For example, the light source(s) positioned to direct light to the binding surface may be activated and the resultant optical signals may be measured by corresponding detector(s). The collection of the sample data may be performed only once the biochemical reaction phase is complete, in some examples. In other examples, the sample data may be collected at multiple time points during interruptions in the biochemical reaction phase. For example, the biochemical reaction phase may be paused so that a first sample dataset may be collected (after a magnetic wash is performed), and then the biochemical reaction phase may be resumed. Then the biochemical reaction phase may be terminated and a second sample dataset may be collected (after another magnetic wash is performed). By collecting sample data at one or more time points before the biochemical reaction phase is completed, signal saturation due to high analyte concentration, for example, may be avoided by measuring the optical signals before the biochemical reaction has gone to completion.

At 1412, the background data and the sample data are optionally corrected based on the reference measurement obtained at 1404. For example, the reference measurement may be subtracted from each of the background data and the sample data. In doing so, other fluctuations that may affect the optical signal (e.g., output from the light sources) may be compensated. At 1414, the sample data is subtracted from the background data to generate corrected background data. As explained above, the optical signals measured during the biochemical reaction phase include signals from both bound and unbound signal generating elements, as the optical signals are measured at the capture element spots (because the background regions overlap the binding surface). Thus, the signals from only the unbound signal generating elements are obtained by removing the optical signals obtained when the biochemical reaction phase is complete, which is indicative of the optical signals of only the bound signal generating elements. The corrected background data may include a separate corrected background dataset for each background region, or the background data may be combined and the combined sample data may be subtracted from the combined background data.

At 1416, the sample data is corrected based on the corrected background data. Correcting the sample data based on the corrected background data may include dividing the sample data by the corrected background data. In other examples, a different function may be applied to correct the sample data using the background data, such as a relation between the sample data and the background data that is established during calibration, where the relation may be linear, a power function, exponential, etc.

At 1418, the corrected sample data may be stored and/or displayed on a display of the sensor system. The corrected sample data may be used to determine a concentration of one or more analytes of interest in the sample, similar to the process described above with respect to FIG. 7, and the determined concentration(s) or concentration signal(s) may be output for display and/or saved in memory. Method 1400 then ends.

While the methods discussed above with respect to FIGS. 7 and 14 are directed to background correction using optical signals obtained at discrete regions, the methods discussed herein could instead rely on images of an entirety of the detection surface of the sample container. For example, during both the biochemical reaction phase and detection phase of a test, images of the entire sample chamber/detection surface may be obtained and stored in memory. Once the detection phase is complete, signals may be extracted from the stored images to obtain the background data (e.g., the signals from unbound magnetic beads during the biochemical reaction phase) and the sample data (e.g., the signals from the bound beads after the biochemical reaction has stopped).

Thus, by measuring the sensor signals from the signal generating elements during the magnetic attraction phase, the combined effects of sample properties and signal generating element distribution inhomogeneities (and possibly other causes) that influence the signal response can be measured and used to directly correct the signal response of the binding surface. By using the signal response from the signal generating elements outside the binding surface areas (e.g., such that the background data is obtained in one or more regions of the detection surface that are not functionalized with capture elements), the background correction is not influenced by the concentration of the analyte(s) being tested, which may influence the signal response within the binding surface also during the magnetic attraction phase (e.g., during this phase, the signal response will increase over time dependent on the analyte concentration). Another advantage of the background correction described herein is that the signals measured outside the binding surface during the biochemical reaction (when magnetic particles are at the binding surface) can be used as a check for the correction functioning of the reaction. For example, if the reaction chamber is not fully filled with liquid but instead contains air inclusions, a signal near zero will be measured at the location of the air inclusion, indicating signal generating elements cannot reach this location area of the detection surface. This information can then be used to invalidate the test and prevent erroneous test results from being released.

The technical effect of correcting sample data indicative of a number of signal generating elements bound to a binding surface based on background data indicative of a number of unbound signal generating elements is that gravitational and fluid composition effects on a concentration of an analyte determined based on the sample data may be compensated, thereby reducing test-to-test variability.

The disclosure also provides support for a sensor system, comprising a sample container configured to receive a sample containing an analyte to be tested, the sample container comprising: a detection surface, and a plurality of signal generating elements in the sample container, wherein the detection surface comprises a binding surface, which has been partially functionalized with capture elements that can bind, directly and/or indirectly, the analyte and/or the plurality of signal generating elements, and a memory storing instructions executable by a processor to: obtain background data comprising sensor signals from one or more background regions of the detection surface, obtain sample data comprising sensor signals from the binding surface, and perform a correction of the sample data based on the background data. In a first example of the system, the one or more background regions of the detection surface are each arranged in an at least partially non-overlapping manner with the binding surface. In a second example of the system, optionally including the first example, the system further comprises: a magnetic element, and wherein the magnetic element is activated to generate a magnetic field to pull the plurality of signal generating elements to the binding surface while the background data is obtained, and the magnetic element is not activated to generate the magnetic field or the magnetic element is activated to keep unbound signal generating elements away from the binding surface while the sample data is obtained. In a third example of the system, optionally including one or both of the first and second examples, at least a portion of signal generating elements of the plurality of signal generating elements includes a capture element that can bind the analyte. In a fourth example of the system, optionally including one or more or each of the first through third examples, the instructions are executable to weight the sensor signals from at least one background region differently than at least one other background region. In a fifth example of the system, optionally including one or more or each of the first through fourth examples, the binding surface comprises a plurality of discrete areas, each area of the binding surface functionalized with the capture elements, and wherein the one or more background regions of the detection surface are each arranged in a non-overlapping manner with the plurality of discrete areas of the binding surface such that each background region is not functionalized with capture elements. In a sixth example of the system, optionally including the one or more or each of first through fifth examples, the plurality of discrete areas of the binding surface is arranged into a first row of areas and a second row of areas, and wherein the one or more background regions of the detection surface comprise a plurality of background regions arranged into a first row of background regions, a second row of background regions, and a third row of background regions. In a seventh example of the system, optionally including one or more or each of the first through sixth examples, the first row of background regions are located proximate a pinning of the sample container, the third row of background regions are located proximate an inlet of the sample container, and the second row of background regions are located intermediate the first row of background regions and the third row of background regions, and wherein the sensor signals for the one or more background regions are weighted such that the sensor signals from the first row of background regions and the third row of background regions are given a higher weight than the sensor signals from the second row of background regions. In an eighth example of the system, optionally including one or more or each of the first through seventh examples, each background region of the detection surface overlaps a respective area of the binding surface. In a ninth example of the system, optionally including one or more or each of the first through eighth examples, the instructions are executable to subtract the sample data from the background data to generate corrected background data, and wherein correcting the sample data based on the background data comprises correcting the sample data based on the corrected background data. In a tenth example of the system, optionally including one or more or each of the first through ninth examples, the instructions are executable to determine a concentration of the analyte in the sample based on the corrected sample data. In an eleventh example of the system, optionally including one or more or each of the first through tenth examples, the instructions are executable to determine the concentration of the analyte in the sample based on the corrected sample data in response to positive, non-zero optical signals being obtained from each of the one or more background regions, and in response to positive, non-zero optical signals not being obtained from each of the one or more background regions, output a notification indicating that the concentration of the analyte cannot be determined.

The disclosure also provides support for a method for a sensor system, comprising during a test of a sample including an analyte contained in a sample container of the sensor system, measuring sensor signals at one or more background regions of a detection surface of the sample container to generate background data, measuring sensor signals at a binding surface of the sample container to generate sample data, wherein the binding surface comprises one or more areas of the detection surface that are functionalized with capture elements that can bind, directly and/or indirectly, the analyte and/or a plurality of signal generating elements of the sample container, and outputting a concentration of the analyte in the sample based on the sample data and the background data. In a first example of the method, the sensor signals at the one or more background regions are measured while the plurality of signal generating elements are being pulled to the binding surface and wherein the sensor signals at the binding surface are measured while the plurality of signal generating elements are not being pulled to the binding surface. In a second example of the method, optionally including the first example, the sensor signals comprise optical signals measured using frustrated total internal reflection.

References to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other.

1. A sensor system, comprising:
a sample container configured to receive a sample containing an analyte to be tested, the sample container comprising:
a detection surface; and
a plurality of signal generating elements in the sample container, wherein the detection surface comprises a binding surface, which has been partially functionalized with capture elements that can bind, directly or indirectly, the analyte or the plurality of signal generating elements; and
a memory storing instructions executable by a processor to:
obtain background data comprising sensor signals from one or more background regions of the detection surface;
obtain sample data comprising sensor signals from the binding surface; and
perform a correction of the sample data based on the background data.

2. The sensor system of claim 1, wherein the one or more background regions of the detection surface are each arranged in an at least partially non-overlapping manner with the binding surface.

3. The sensor system of claim 1, further comprising a magnetic element, and wherein the magnetic element is activated to generate a magnetic field to pull the plurality of signal generating elements to the binding surface while the background data is obtained, and the magnetic element is not activated to generate the magnetic field or the magnetic element is activated to keep unbound signal generating elements away from the binding surface while the sample data is obtained.

4. The sensor system of claim 1, wherein at least a portion of signal generating elements of the plurality of signal generating elements includes a capture element that can bind the analyte.

5. The sensor system of claim 1, wherein the instructions are executable to weight the sensor signals from at least one background region differently than at least one other background region.

6. The sensor system of claim 1, wherein the binding surface comprises a plurality of discrete areas, each area of the binding surface functionalized with the capture elements, and wherein the one or more background regions of the detection surface are each arranged in a non-overlapping manner with the plurality of discrete areas of the binding surface such that each background region is not functionalized with capture elements.

7. The sensor system of claim 6, wherein the plurality of discrete areas of the binding surface is arranged into a first row of areas and a second row of areas, and wherein the one or more background regions of the detection surface comprise a plurality of background regions arranged into a first row of background regions, a second row of background regions, and a third row of background regions.

8. The sensor system of claim 7, wherein the first row of background regions are located proximate a pinning of the sample container, the third row of background regions are located proximate an inlet of the sample container, and the second row of background regions are located intermediate the first row of background regions and the third row of background regions, and wherein the sensor signals for the one or more background regions are weighted such that the sensor signals from the first row of background regions and the third row of background regions are given a higher weight than the sensor signals from the second row of background regions.

9. The sensor system of claim 1, wherein each background region of the detection surface overlaps a respective area of the binding surface.

10. The sensor system of claim 1, wherein the instructions are executable to subtract the sample data from the background data to generate corrected background data, and wherein correcting the sample data based on the background data comprises correcting the sample data to generate corrected sample data based on the corrected background data.

11. The sensor system of claim 10, wherein the instructions are executable to determine a concentration of the analyte in the sample based on the corrected sample data.

12. The sensor system of claim 11, wherein the instructions are executable to determine the concentration of the analyte in the sample based on the corrected sample data in response to positive, non-zero optical signals being obtained from each of the one or more background regions, and in response to positive, non-zero optical signals not being obtained from each of the one or more background regions, output a notification indicating that the concentration of the analyte cannot be determined.

13. A method for a sensor system, comprising:
during a test of a sample including an analyte contained in a sample container of the sensor system, measuring sensor signals at one or more background regions of a detection surface of the sample container to generate background data;
measuring sensor signals at a binding surface of the sample container to generate sample data, wherein the binding surface comprises one or more areas of the detection surface that are functionalized with capture elements that can bind, directly or indirectly, the analyte or a plurality of signal generating elements of the sample container; and
outputting a concentration of the analyte in the sample based on the sample data and the background data.

14. The method of claim 13, wherein the sensor signals at the one or more background regions are measured while the plurality of signal generating elements are being pulled to the binding surface and wherein the sensor signals at the binding surface are measured while the plurality of signal generating elements are not being pulled to the binding surface.

15. The method of claim 13, wherein the sensor signals comprise optical signals measured using frustrated total internal reflection.
